(19) United States
(12) Patent Application Publication
(10) Pub. No.: US 2023/0091009 A1
(43) Pub. Date: Mar. 23, 2023

(54) METHOD FOR PROMOTING GROWTH OF GAS-FERMENTED MICROORGANISMS

(71) Applicant: HARBIN INSTITUTE OF TECHNOLOGY, SHENZHEN, Shenzhen (CN)

(72) Inventors: LU LU, Shenzhen (CN); Yongjie Yu, Shenzhen (CN); Ruijie Yang, Shenzhen (CN)

(21) Appl. No.: 17/896,103

(22) Filed: Aug. 26, 2022

(30) Foreign Application Priority Data
Sep. 17, 2021 (CN) 202111091233.3

(51) Int. Cl. C12N 1/20; C12N 1/38

(52) U.S. Cl. CPC C12N 1/20; C12N 1/38; C12N 2500/02; C12N 2500/42; C12N 2500/72

(57) ABSTRACT

The present invention relates to a method for promoting growth of gas-fermented microorganisms, comprising the following steps: S1. ultrasonically blending a surfactant and a culture medium, then adding a fluorine-containing alkyl compound to a mixture, and ultrasonically processing the mixture to obtain a perfluorocarbon nanoemulsion; S2. inoculating a bacterial suspension into the perfluorocarbon nanoemulsion, and introducing simultaneously a mixed gas to obtain a precursor; S3. cultivating the precursor in a shaker, wherein the precursor has a diameter of bubbles of 2.0-4.2 mm, and the bubbles have the total volume of less than 40 ml. Under these conditions, the microorganisms have a high utilization rate of the gas, can grow and metabolize more quickly, and can obtain bacteria and products more quickly. Compared with a traditional culture system, addition of a mass transfer material can promote gas mass transfer. Therefore, the gas consumption is small, and the cost is low.

### TECHNICAL FIELD

The present invention belongs to the field of microorganism culture, and particularly relates to a method for promoting growth of gas-fermented microorganisms.

### BACKGROUND

With the continuous exploration of waste recycling by cutting-edge technologies, people's perception of sewage has gradually changed from “waste to be treated” to “a carrier of a resource”. A large amount of organic pollutants is contained in sewage. If a Chemical Oxygen Demand (COD) of typical domestic sewage is assumed to be 500 mg/l, the potential energy contained in the typical domestic sewage is 17.7-28.7 kJ/g COD. Standard-concentration sewage has an actual processing energy demand of about 0.45 kW h/m³, which is equivalent to a COD of 3.20 kJ/g. This shows that organic chemical energy contained in the sewage is nearly 5 times the energy consumption required for treatment for the sewage, which has great application potential.

Using microbial electrochemistry to produce hydrogen from sewage treatment or anaerobic fermentation of activated sludge may obtain hydrogen energy while removing pollutants, which may realize waste recycling and energization. Based on renewable energy electric auxiliary hydrogen production/bio-electrocatalytic hydrogen production/bio-electric stimulation hydrogen production, an organic matter/energy in sewage can be recycled and utilized. At the same time, gases such as carbon dioxide, methane, and hydrogen are produced. Excessive emissions of methane and carbon dioxide that are greenhouse gases aggravate climate changes. Extreme weather continues to occur around the world, causing huge economic losses. China has formulated a goal of energy conservation and emission reduction, which requires carbon emission reduction and carbon dioxide fixation. Anaerobic fermentation gas production and microbial electrochemical hydrogen production have disadvantages of low gas purity and existence of carbon dioxide and other gases, which has low value. Therefore, it is difficult to use in the next step. Secondly, on the one hand, some industrial production processes, such as petroleum refining, steelmaking, ammonia synthesis, coal-to-methanol, etc., can emit a large amount of waste gas, mainly carbon dioxide and hydrogen, into the atmosphere directly or through combustion. On the other hand, wood fibers those are difficult to be biodegraded can be firstly converted into a syngas by a thermochemical method, that is, a mixture of hydrogen, carbon monoxide and carbon dioxide, and then further processed to realize the utilization of resources.

Gas fermentation is to use metabolic activities of microorganisms to synthesize organic substances while meeting requirements of growth of the microorganisms with gas as a substrate. Gas-fermented microorganisms refer to autotrophic microorganisms that use gas as an energy material for metabolic activities. In a process of synthetic fermentation, such microorganisms need an energy material (an electron donor) and a carbon source, such as hydrogen, carbon monoxide, methane, etc. Anaerobic fermentation gas production, synthesis gas and microbial electrochemical hydrogen production can just meet needs of gas-fermented microorganisms, and can achieve an objective of using clean energy to biodegrade pollutants in the sewage and produce a high-value drug/fuel/protein.

A traditional use of hydrogen and other gases is mainly for combustion and chemical synthesis. For example, hydrogen is used as a raw material for synthesis for ammonia, methanol, and hydrochloric acid; a metallurgical reducing agent, and a hydrodesulfurization agent in petroleum refining. Methane can be used for synthesis of ammonia, urea and carbon black and can also be used for synthesis of ethylene, formaldehyde, carbon disulfide, nitromethane, hydrocyanic acid, and 1,4-butanediol. Compared with chemical treatment of these gases, biocatalysts have the advantages of mild reaction conditions, high reaction specificity, high tolerance to sulphide, and no need to adjust a proportion of a specific gas, which have received special attention in recent years. However, under ambient conditions, solubility of the syngas in water is very limited. For example, hydrogen, even in a supersaturated state, has solubility of only about 0.79 mmol/l. Therefore, low gas mass transfer efficiency has been a bottleneck in a fermentation process of the syngas and production of high-value chemicals.

Perfluorocarbon, also known as a perfluorinated solvent or a fluorine solvent, is alkane, ether and amine in which all hydrogen atoms on carbon atoms are replaced by fluorine atoms. In existing reports, the perfluorocarbon is an excellent gas solvent, which can dissolve a large amount of hydrogen, oxygen, nitrogen and carbon dioxide. The literature (Dissolving Gases in FLUTEC Liquids (F2 Chemicals Ltd, 2005)) reported that solubility of hydrogen and carbon dioxide in the perfluorocarbon is increased by an order of magnitude compared to directly dissolving the gas in an aqueous solution. Moreover, the perfluorocarbon is chemically inert, and is not actually combined with gas molecules directly in a process of dissolving gas, showing an excellent role as a gas carrier. However, the pure perfluorocarbon has a very high density ranging from 1.7 to 2.0 g/cm³. When applied to a system with a low shear and mild mixture, the perfluorocarbon is either completely sunk to a bottom or only partially dispersed into large droplets, resulting in a very low enhancement efficiency for a gas mass transfer effect.

Also, mass transfer between a gas and a liquid is affected by a gas diffusion coefficient. Based on Lemlich model and by constructing an empirical formula, it can be seen that a gas diffusion coefficient is affected by a diameter of the bubbles and the stability of the bubbles. Stable bubbles with a small diameter have a small gas diffusion coefficient. The bubbles with a small gas mass transfer coefficient block contact between the liquid and the gas, thereby inhibiting the mass transfer of the gas.

Therefore, there is an urgent need to find a technology to overcome the forgoing shortcomings of the perfluorocarbon in culturing the growth of the gas-fermented microorganisms.

### SUMMARY

In view of the forgoing technical problems, the present invention discloses a method for promoting growth of gas-fermented microorganisms, which can effectively improve gas mass transfer efficiency, thereby promoting a fermentation process of a syngas to proceed more smoothly. The method is mainly to inoculate a bacterial suspension into a perfluorocarbon emulsion, and at the same time, a mixed gas is introduced. Therefore, the gas-fermented microorganisms are continuously cultivated in this environment. Surprisingly, it was found that this method can greatly promote the growth of the gas-fermented microorganisms, thereby greatly speeding up synthesis of an organic matter.

A perfluorocarbon aqueous solution is ultrasonically treated with a biocompatible polymer surfactant containing an alkoxy chain segment. A perfluorocarbon nanoemulsion is prepared by emulsification, so that the perfluorocarbon is dispersed in an aqueous solution in a form of nano-scale particles. Compared with a pure perfluorocarbon solvent, the perfluorocarbon nanoemulsion has a smaller nano-scale particle size, a larger liquid-liquid interface area, and a non-specific bond formed with microorganisms, which can greatly improve solubility of the syngas in a microbial fermentation culture system, improve the gas mass transfer efficiency and overcome a bottleneck of the fermentation of the syngas.

The present invention aims to provide a method for promoting growth of gas-fermented microorganisms, which is achieved through the following technical solutions.

A method for promoting growth of gas-fermented microorganisms, comprising the following steps:

S1. ultrasonically blending a surfactant and a culture medium, then adding a fluorine-containing alkyl compound to a mixture, and ultrasonically processing the mixture to obtain a perfluorocarbon nanoemulsion;

S2. inoculating a bacterial suspension into the perfluorocarbon nanoemulsion, and introducing simultaneously a mixed gas to obtain a precursor;

S3. cultivating the precursor in a shaker;

The bubbles in the precursor have a diameter of 2-4.2 mm, and the total volume of less than 40 ml.

In some embodiments of the present invention, the specific diameter of the bubbles may be, but not limited to, 2 mm, 2.5 mm, 3 mm, 3.5 mm, 4 mm and 4.2 mm. A total volume of the bubbles may be, but not limited to, 40 ml, 35 ml, 30 ml, 25 ml and 20 ml.

During preparation of the forgoing precursor, the perfluorocarbon nanoemulsion, as a surfactant, forms many bubbles under conditions of sufficient shaking. After the bacterial suspension is inoculated, the microorganisms are cultured in a closed system. The mixed gas is mainly concentrated in an upper headspace. Therefore, the existence of the bubbles has a significant impact on gas mass transfer and the growth of microorganisms. When the perfluorocarbon nanoemulsion is added and the bubbles have a larger diameter, the mass transfer effect is good. Therefore, a precursor environment can promote microbial growth and increase a product yield. Otherwise, an inhibitory effect is provided. Secondly, the bubbles separate an upper gas and a liquid surface. The smaller the total volume of the bubbles, the easier the contact between the gas and the liquid. Therefore, the mass transfer of the gas is promoted. Therefore, the bubbles have an ideal state of the total volume of less than 40 ml.

The diameter of the bubbles is related to a liquid flow rate and a diameter of an aeration hole. The larger the liquid flow rate, the smaller the diameter of the bubbles. The smaller the aeration hole, the smaller the diameter of the bubbles. When the perfluorocarbon nanoemulsion is added, vigorous shaking is avoided. A container with a large diameter (greater than 2 mm) is used to transfer the perfluorocarbon nanoemulsion slowly, resulting in a small number of bubbles with a large diameter. the perfluorocarbon nanoemulsion was added by a syringe with a needle (a diameter of 0.6 mm) quickly. Vigorously shaking is performed to form a larger number of bubbles with the smaller diameter upon injection, thus forming an ideal state with the bubbles with the diameter of 2.0-4.2 mm.

Further, the mixed gas is selected from one or more of nitrogen, argon, oxygen, carbon dioxide, carbon monoxide and hydrogen.

Further, the compositions of the culture medium comprise one or more of halogenide, phosphate, hydrophosphate, sulphate, and sulphite, hydrates of the forgoing compositions, and trace elements.

Further, the bacterial suspension has an OD600 value of 0.05-1.

Further, the surfactant is selected from a polymer surfactant containing an alkoxy chain segment.

Further, the fluorine-containing alkyl compound is selected from one or more of perfluorodecalin, tetradecafluorohexane, dodecafluorocyclohexane, perfluoroheptane, dodecafluoropentane, decafluoropentane, and heptafluoropropane.

Further, the perfluorocarbon nanoemulsion has an average particle size of 200-300 nm.

Further, a bacterium is cupriavidus necator.

The cupriavidus necator is inoculated in the perfluorocarbon nanoemulsion. A specific air pressure can effectively improve the growth efficiency of the microorganisms, at the same time, play a role of biological carbon fixation, and obtain a high-value metabolite poly-β-hydroxybutyric acid (PHB).

Further, the culture medium has a temperature of 30-37° C., a rotational speed of 100-300 rpm; time of 48-120 h and pH of 6.5-7.5.

Further, the mixed gas has a percent by volume of hydrogen of 20-50 vt %.

Preferably, the mixed gas includes hydrogen and oxygen to ensure that the microorganisms can perform aerobic respiration and have sufficient energy substances. Preferably, a pressure generated by the introduced mixed gas is 100-200 kPa.

The present invention has the following beneficial effects:

1. Under these conditions, the microorganisms have a high utilization rate of the gas, may grow and metabolize more quickly, and may obtain bacteria and products more quickly.
2. Compared with a traditional culture system, addition of a mass transfer material can promote gas mass transfer. Therefore, the gas consumption is small, and the cost is low.

### BRIEF DESCRIPTION OF THE DRAWINGS

FIG. 1 shows an average particle size distribution diagram of a perfluorocarbon nanoemulsion of Embodiment 1.

FIGS. 2A-2C show an appearance of cultured precursors of Embodiment 1 and Comparative Embodiments 1-2, respectively.

FIG. 3 shows the OD600 value and PHB produced in Embodiment 1, Comparative Embodiments 1-2.

### DETAILED DESCRIPTION OF EMBODIMENTS

In order to illustrate the technical solutions of the present invention more clearly, the following embodiments are given. Unless otherwise stated, the raw materials, reactions and post-processing means in the embodiments are common raw materials on market and technical means well known to those skilled in the art.

Bacteria described in embodiments of the present invention adopt cupriavidus necator.

A surfactant described in the embodiments of the present invention is a polymer surfactant containing an alkoxy chain segment, which is Pluronic series products purchased from BASF.

A method for testing a particle size of a perfluorocarbon nanoemulsion described in the embodiments and comparative embodiments of the present invention uses a nanoparticle size and a surface potential tester for measurement.

The method for testing a diameter of bubbles described in the embodiments and the comparative embodiment of the present invention adopts ImageJ software to measure a real picture.

The method for testing a total volume of the bubbles described in the embodiments and the comparative embodiment of the present invention adopts ImageJ software to measure the real picture.

pH of a culture medium described in Embodiments 1-3 of the present invention is 6.8, and the compositions and concentration of the culture medium are described in Table 1 below.

TABLE 1
Compositions and Concentration of Culture Medium

| Compositions | Concentration (g/l) |
|---|---|
| NH₄C1 | 1.0 |
| NaH,PO4•2H₂O | 5.2 |
| Na₂HPO₄•12H₂O | 11.6 |
| K₂SO₄ | 0.45 |
| MgSO₄•7H₂O | 0.80 |
| CaCl₂•2H₂O | 0.08 |
| Trace elements | 0.001 |

The forgoing trace elements as well as the compositions and concentrations thereof were described in Table 2 below.

TABLE 2
Composition and Concentration of Trace Elements

| Compositions | Concentration (g/l) |
|---|---|
| HCl | 0.1 |
| FeSO₄•7H₂O | 15 |
| MnSO₄•H₂O | 2.4 |
| ZnSO₄•7H₂O | 2.4 |
| CuSO₄•5H₂O | 0.48 |

### Embodiment 1

A method for promoting growth of gas-fermented microorganisms, comprising the following steps:

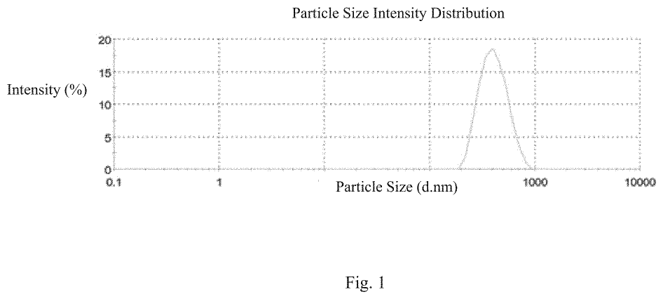

S1. ultrasonically blending a surfactant Pluronic F68 and 50 ml of a culture medium (wherein, Pluronic F68 is 2.8 wt % of the culture medium) with an ultrasonic cleaner for 30 min, so that the surfactant was completely dissolved; taking 2250 μl of perfluorodecalin and 2250 μl of tetradecafluorohexane, ultrasonically blending with an ultrasonic cleaner for 30 min to obtain a perfluorocarbon nanoemulsion, the perfluorocarbon nanoemulsion at this time having a measured average particle size of about 254 nm, and obtained particle size test results being shown in FIG. 1;

S2. culturing a cupriavidus necator with a culture medium for 20 h to obtain an intermediate, then centrifuging, pouring out a supernatant, washing with 25 ×PBS, and then pouring out the supernatant by centrifugation. This step was repeated three times, and bacteria were dispersed in the culture medium to obtain a bacterial suspension.

Establishing 2 groups of parallel experiments: covering an anaerobic bottle with a rubber stopper and an aluminum seal, sterilizing in a sterilizer at 121° C. for 20 min, adding the perfluorocarbon nanoemulsion to the bacterial suspension, mixing evenly, then inoculating 45 ml of the bacterial suspension containing the perfluorocarbon nanoemulsion into the anaerobic bottle, introducing a mixed gas (a volume ratio of each gas in the mixed gas was N₂:H₂:O₂:CO₂=49:37:7:7) at the same time, making a pressure in the anaerobic bottle be about 0.3 MPa to obtain a precursor, the bubbles in the precursor having the diameter of 2 mm, and the bubbles having the total volume of 30 ml.

S3. putting the precursor into a shaker, and culturing at a constant temperature of 30° C., at a rotation speed of 100 rpm, and at pH of 6.5 for 72 h.

### Embodiment 2

A method for promoting growth of gas-fermented microorganisms, comprising the following steps:

S1. ultrasonically blending a surfactant Pluronic F68 and 30 ml of a culture medium (wherein, Pluronic F68 is 2.9 wt % of the culture medium) with an ultrasonic cleaner for 30 min, so that the surfactant was completely dissolved; taking 1350 μl of perfluorodecalin and 1350 μl of tetradecafluorohexane, ultrasonically blending with an ultrasonic cleaner for 20 min to obtain a perfluorocarbon nanoemulsion, the perfluorocarbon nanoemulsion at this time having a measured average particle size of about 280 nm.

S2. culturing a cupriavidus necator with a culture medium for 22 h to obtain an intermediate, then centrifuging, pouring out a supernatant, washing with 25×PBS, and then pouring out the supernatant by centrifugation. This step was repeated three times, and bacteria were dispersed in the culture medium to obtain a bacterial suspension.

Establishing 2 groups of parallel experiments: covering an anaerobic bottle with a rubber stopper and an aluminum seal, sterilizing in a sterilizer at 121° C. for 20 min, adding the perfluorocarbon nanoemulsion to the bacterial suspension, mixing evenly, then inoculating 50 ml of the bacterial suspension containing the perfluorocarbon nanoemulsion into the anaerobic bottle, introducing a mixed gas (a volume ratio of each gas in the mixed gas was N₂:H₂:O₂:CO₂=40:40:10:10) at the same time, making a pressure in the anaerobic bottle be about 0.3 MPa to obtain a precursor, the bubbles in the precursor having the diameter of 4.2 mm, and the bubbles having the total volume of 40 ml.

S3. putting the precursor into a shaker, and culturing at a constant temperature of 32° C., at a rotation speed of 300 rpm, and at pH of 7.5 for 96 h.

### Embodiment 3

A method for promoting growth of gas-fermented microorganisms, comprising the following steps:

S1. ultrasonically blending a surfactant Pluronic F68 and 20 ml of a culture medium (wherein, Pluronic F68 is 3.0 wt % of the culture medium) with an ultrasonic cleaner for 30 min, so that the surfactant was completely dissolved; taking 900 μl of perfluorodecalin and 900 μl of tetradecafluorohexane, ultrasonically blending with an ultrasonic cleaner for 35 min to obtain a perfluorocarbon nanoemulsion, the perfluorocarbon nanoemulsion at this time having a measured average particle size of about 250 nm.

S2. culturing a cupriavidus necator with a culture medium for 20 h to obtain an intermediate, then centrifuging, pouring out a supernatant, washing with 25×PBS, and then pouring out the supernatant by centrifugation. This step was repeated three times, and bacteria were dispersed in the culture medium to obtain a bacterial suspension.

Establishing 2 groups of parallel experiments: covering an anaerobic bottle with a rubber stopper and an aluminum seal, sterilizing in a sterilizer at 121° C. for 20 min, adding the perfluorocarbon nanoemulsion to the bacterial suspension, mixing evenly, then inoculating 45 ml of the bacterial suspension containing the perfluorocarbon nanoemulsion into the anaerobic bottle, introducing a mixed gas (a volume ratio of each gas in the mixed gas was N₂:H₂:O₂:CO₂=45:40:8:7) at the same time, making a pressure in the anaerobic bottle be about 0.4 MPa to obtain a precursor, the bubbles in the precursor having the diameter of 3.5 mm, and the bubbles having the total volume of 35 ml.

S3. putting the precursor into a shaker, and culturing at a constant temperature of 35° C., at a rotation speed of 200 rpm, and at pH of 7.0 for 120 h.

### Comparative Embodiment 1

S1. culturing a cupriavidus necator with a culture medium for 20 h to obtain an intermediate, then centrifuging, pouring out a supernatant, washing with 25×PBS, and then pouring out the supernatant by centrifugation. This step was repeated three times, and bacteria were dispersed in the culture medium to obtain a bacterial suspension.

Establishing 2 groups of parallel experiments: covering an anaerobic bottle with a rubber stopper and an aluminum seal, sterilizing in a sterilizer at 121° C. for 20 min, then inoculating 45 ml of the bacterial suspension into the anaerobic bottle, introducing a mixed gas (compositions of each gas in the mixed gas was N₂:H₂:O₂:CO₂=49:37:7:7) at the same time, and making a pressure in the anaerobic bottle be about 0.3 MPa to obtain a precursor.

S2. putting the precursor into a shaker, and culturing at a constant temperature of 30° C., at a rotation speed of 100 rpm, and at pH of 6.5 for 72 h. No air bubbles are provided in the precursor.

### Comparative Embodiment 2

Substances and an operation method used in Comparative Embodiment 2 were the same as those in Embodiment 1. Only difference was that during preparation of the precursor, a shaking force of the perfluorocarbon nanoemulsion was slightly increased, so that the bubbles in the precursor had a diameter of 1.1 mm, and a total volume of 60 ml.

### Test Embodiments

An anaerobic bottle of a cultured precursor of Embodiment 1 and Comparative Embodiments 1-2 was opened. An OD600 value measured by a bacterial liquid was used to characterize growth of microorganisms. 2 ml of the bacterial liquid was centrifuged and dissolved simultaneously. A liquid chromatograph was used to measure PHB to characterize a product yield.

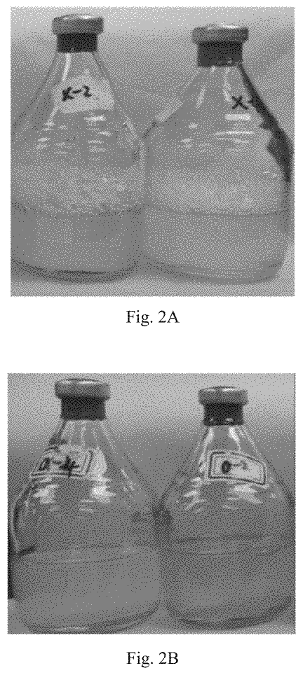

FIGS. 2A-2C showed an appearance of cultured precursors of Embodiment 1 and Comparative Embodiments 1-2, respectively. As can be seen from FIGS. 2a-c, a diameter and a total volume of bubbles in Embodiment 1 were moderate. In Comparative Embodiment 1, since no surfactant was added, no bubbles were generated. In Comparative Embodiment 2, due to addition of a surfactant, vigorously shaking was performed. Therefore, the bubbles had a smaller diameter and the largest total volume.

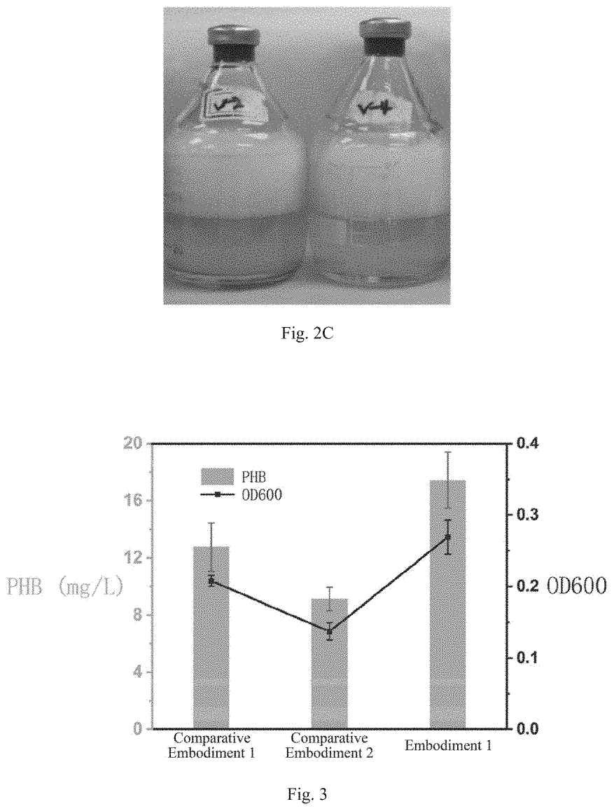

FIG. 3 showed measured OD600 and PHB results of the cultured precursor of Embodiment 1 and Comparative Embodiments 1-2.

As can be seen from FIG. 3, in Embodiment 1, the surfactant was added. The bubbles were controlled to be moderate in the diameter. Growth of microorganisms and a yield of a product PHB were the best. No surfactant was added in Comparative Embodiment 1. The PHB yield under normal growth of the microorganisms was 12.8 mg/l. However, in Comparative Embodiment 2, due to the small diameter and the excessive total volume of the bubbles, a mass transfer coefficient was small, which greatly hindered the growth of the microorganisms and the synthesis of the product.

It will be apparent to those skilled in the art that the present invention is not limited to the details of the forgoing exemplary embodiments, but that the present invention may be embodied in other specific forms without departing from the spirit or essential characteristics of the present invention. Therefore, the embodiments are to be regarded in all respects as illustrative and not restrictive. The scope of the present invention is defined by the appended claims rather than the foregoing description. All changes within the meaning and scope of the equivalents of claims are included in the present invention.

In addition, it should be understood that although this specification is described in terms of embodiments, not each embodiment only includes an independent technical solution. This description in the specification is only for the sake of clarity. Those skilled in the art should take the specification as a whole. The technical solutions in each embodiment can also be appropriately combined to form other implementations that can be understood by those skilled in the art.

1. A method for promoting growth of gas-fermented microorganisms, comprising the following steps:
S1. ultrasonically blending a surfactant and a culture medium, then adding a fluorine-containing alkyl compound to a mixture, and ultrasonically processing the mixture to obtain a perfluorocarbon nanoemulsion;
S2. inoculating a bacterial suspension into the perfluorocarbon nanoemulsion, and introducing simultaneously a mixed gas to obtain a precursor;
S3. cultivating the precursor in a shaker;
wherein, bubbles in the precursor have a diameter of 2-4.2 mm, and the total volume of less than 40 ml.

2. The method for promoting the growth of the gas-fermented microorganisms according to claim 1, wherein the mixed gas is selected from one or more of nitrogen, argon, oxygen, carbon dioxide, carbon monoxide and hydrogen.

3. The method for promoting the growth of the gas-fermented microorganisms according to claim 1, wherein the compositions of the culture medium comprise one or more of halogenide, phosphate, hydrophosphate, sulphate, and sulphite, hydrates of the forgoing compositions, and trace elements.

4. The method for promoting the growth of the gas-fermented microorganisms according to claim 1, wherein the bacterial suspension has an OD600 value of 0.05-1.

5. The method for promoting the growth of the gas-fermented microorganisms according to claim 1, wherein the surfactant is selected from a polymer surfactant containing an alkoxy chain segment.

6. The method for promoting the growth of the gas-fermented microorganisms according to claim 1, wherein the fluorine-containing alkyl compound is selected from one or more of perfluorodecalin, tetradecafluorohexane, dodecafluorocyclohexane, perfluoroheptane, dodecafluoropentane, decafluoropentane, and heptafluoropropane.

7. The method for promoting the growth of the gas-fermented microorganisms according to claim 1, wherein the perfluorocarbon nanoemulsion has an average

8. The method for promoting the growth of the gas-fermented microorganisms according to claim 1, wherein a bacterium is cupriavidus necator.

9. The method for promoting the growth of the gas-fermented microorganisms according to claim 1, wherein the culture medium has a temperature of 30-37° C., a rotational speed of 100-300 rpm; time of 48-120 h and pH of 6.5-7.5.

10. The method for promoting the growth of the gas-fermented microorganisms according to claim 2, wherein the mixed gas has a percent by volume of hydrogen of 20-50 vt %.
